(19) United States
(12) Patent Application Publication
(10) Pub. No.: US 2022/0313734 A1
(43) Pub. Date: Oct. 6, 2022

(54) FIBROBLAST GENERATED PATIENT-SPECIFIC VACCINES

(71) Applicant: Figene, LLC, Houston, TX (US)

(72) Inventors: Pete O'Heeron, Houston, TX (US); Thomas lchim, San Diego, CA (US)

(21) Appl. No.: 17/595,112

(22) PCT Filed: May 8, 2020
(86) PCT No.: PCT/US2020/032207

§ 371 Date: Nov. 9, 2021

Related U.S. Application Data
(60) Provisional application No. 62/845,403, filed on May 9, 2019.

(51) Int. Cl. A61K 35/13; A61K 35/33; A61P 35/00; C12N 5/079

(52) U.S. Cl. CPC A61K 35/13; A61K 35/33; A61P 35/00; C12N 5/0618; C12N 2501/11; C12N 2506/1307; C12N 2533/52

(57) ABSTRACT

The disclosure includes embodiments for utilizing fibroblasts derived from cancer patients and generating “de novo” tumor specific cancer cells and cancer stem cells. The cells may be used as a source of one or more patient-specific antigens for generating one or more personalized tumor vaccines.

This application claims priority to U.S. Provisional Patent Application Ser. No. 62/845,403, filed May 9, 2019, which is incorporated by reference herein in its entirety.

### TECHNICAL FIELD

The disclosure concerns at least the fields of cell biology, molecular biology, immunology, and medicine, including cancer medicine.

### BACKGROUND

Treatment of neoplasia using the body's own natural protective mechanisms has been described as “Breakthrough of the Year” in light of positive data generated utilizing checkpoint inhibitors, as well as chimeric antigen receptor (CAR) T cells. Unfortunately, response rates still are between 10-30%, with some tumor types not responding.

While it is intellectually appealing to augment cancer specific immunity, a draw-back of cancer vaccination is the potential to augment or accelerate tumor growth in response to the vaccine. For example, Flexner and Jobling showed that injection of dead autologous tumor cells enhanced the growth of pre-existing tumors [1]. In general, Th2-driven antibody responses to tumors are non-protective and may contribute to tumor progression by inhibiting the Th1 cell-mediated immune response. It may be that this occurs because of non-useful adjuvants being administered that stimulate Th2 responses as compared to Th1, which are known to induce cytotoxic antibodies [2]. Kaliss popularized the term “immunological enhancement” to describe the enhancement of tumor growth by non-cytotoxic antibodies [3]. It was theorized that these antibodies bind to tumor cells, masking their epitopes and thus preventing a cell-mediated immune response, although this has never been demonstrated experimentally. This is similar to the theory of immunostimulation of tumor growth, which states that, in contrast to the strong immune response generated by transplantable tumors, a quantitatively mild immune response, such as that generated by spontaneous tumors, is stimulatory to the growth of neoplasia [4]. Several experimental observations support the hypothesis that such a weak immune response to cancer may stimulate tumor growth. The co-injection of lymphocytes (spleen cells) from syngeneic mice that had been growing tumors for 10-20 days with tumor cells from MCA-induced sarcomas into thymectomized irradiated syngeneic mice at a range of doses accelerated tumor growth when the ratio of lymphocytes to tumor cells was low. However, when the ratio of lymphocytes to tumor cells was high, lymphocytes from specifically immunized mice inhibited growth compared with naïve lymphocytes that continued to augment tumor growth. This suggests the existence of a biphasic dose response whereas a “weak” immune response results in stimulation of tumor growth while a strong immune response results in protection [4]. One evidence for enhancement of tumor growth in response to vaccination is provided by cancer vaccine clinical trials in which vaccination augments tumor relapse [5].

The utilization of antigen-specific immune stimulation is potentially superior to antigen-nonspecific approaches, such as checkpoint inhibitors. When checkpoint inhibitors are used clinically, latent T cell clones are activated to proliferate. While this includes tumor specific T cells, that are generally repressed by tumors, this also includes autoreactive T cells. This explains the higher incidence of toxicities associated with autoimmunity in patients receiving checkpoint inhibitors. It has been reported that up to 20% of patients receiving checkpoint inhibitors have some degree of autoimmunity, most prevalently colitis. Given the recent introduction of checkpoint inhibitors into widespread clinical use, it may be that autoimmunity may develop in cancer patients during analysis of extended follow-up.

There is a need for developing patient-specific vaccination strategies. While numerous tumor antigens exist, the specific combination of the antigens on patient tumors widely varies. The disclosure encompasses the generation of tumors from patient-specific starting materials at least for the purpose of generating one or more personalized tumor vaccines

### BRIEF SUMMARY

The disclosure encompasses cancer immunological compositions (including vaccines) and methods for inducing immune responses to an individual's own tumors, for example using a patient-specific immunotherapy. In particular embodiments, the disclosure pertains to the field of training the immune system of an individual to kill cancer cells. In a specific case, cells from the same individual are utilized to generate a therapeutic immunological composition against cancer.

Methods of the disclosure utilize inducible pluripotent stem cell technology to generate replicas of cancer that are inactivated and that serve as a trigger to stimulate an immune response to kill cancer cells in an individual, including in primary tumor and/or metastatic tumors of the individual.

In embodiments of the disclosure, there is a method of preparing an immunological composition for cancer for an individual, comprising the steps of: (a) generating pluripotent-like cells from fibroblasts; and (b) performing one or both of the following: (1) exposing the pluripotent-like cells to one or more differentiation factors that differentiate the pluripotent-like cells to neoplastic-like cells; and/or (2) exposing the pluripotent-like cells to one or more mutagenic agents, thereby producing neoplastic-like cells; wherein the neoplastic-like cells and/or derivatives and/or lysates thereof are comprised in the immunological composition and/or are used as an antigenic source for antigen presenting cells for the individual. The neoplastic-like cells may or may not be expanded in culture prior to a use. The culture may or may not comprise feeder cells, such as fibroblast cells.

In specific embodiments, the pluripotent-like cells or the neoplastic-like cells are differentiated into cells having one or more markers of the same tissue as the tissue of the cancer. The neoplastic-like cells and/or derivatives and/or lysates thereof may be exposed (such as ex vivo) to dendritic cells to produce antigen-loaded dendritic cells. The exposure of the lysate and/or cell fragments to dendritic cells may or may not occur in the presence of one or more dendritic cell activators. The antigen-loaded dendritic cells may or may not be co-cultured with T lymphocytes to produce antigen-specific T cells. In some cases, the dendritic cells and the fibroblast cells are from the same individual.

Any pluripotent-like cells may be generated from fibroblasts upon exposure of the fibroblasts to NANOG; OCT-4; SOX-2; any type of stem cells and/or cytoplasm from stem cells; one or more histone deacetylase inhibitors; one or more DNA methyltransferase inhibitors; one or more histone modifiers; umbilical cord blood serum; one or more GSK-3 inhibitors; or a combination thereof. The pluripotent-like cells may be generated from fibroblasts upon exposure of the fibroblasts to reversin, cord blood serum, lithium, a GSK-3 inhibitor, resveratrol, pterostilbene, selenium, (−)-epigallocatechin-3-gallate (EGCG), valproic acid and/or salts of valproic acid, or a combination thereof. Examples of histone deacetylase inhibitors include the following: a) valproic acid; b) sodium phenylbutyrate; c) butyrate; d) trichostatin A; and e) a combination thereof. In some cases, the DNA methyltransferase inhibitor is selected from the group consisting of a) decitabine; b) 5-azacytidine; c) Zebularine; d) RG-108; e) procaine hydrochloride; f) Procainamide hydrochloride; g) Hydralazine hydrochloride; h) Epigallocatechin gallate; i) Chlorogenic acid; j) Caffeic acid; and h) a combination thereof. Any de-differentiated fibroblasts may be exposed to 2%-8%, 2%-7%, 2%-6%, 2%-5%, 2%-4%, 2%-3%, 3%-8%, 3%-7%, 3%-6%, 3%-5%, 3%-4%, 4%-8%, 4%-7%, 4%-6%, 4%-5%, 5%-8%, 5%-7%, 5%-6%, 6%-8%, 6%-7%, or 7%-8% oxygen.

In some embodiments, one or more of the following occurs: (a) an effective amount of the immunological composition is provided to an individual; (b) an effective amount of antigen-loaded dendritic cells produced upon exposure of dendritic cells to lysate and/or cell fragments from the neoplastic-like cells are provided to an individual; (c) an effective amount of antigen-specific T cells produced upon exposure of the antigen-loaded dendritic cells to T lymphocytes are provided to an individual. In specific cases one or more adjuvants (one or more toll like receptors) may be also provided to the individual in (a), (b), or (c). One or more tumor endothelial antigens may be provided to the individual in (a), (b), or (c). One or more tumor endothelial antigens may be selected from the group consisting of Flt-3 ligand, TEM-1, NANOG, SOX2, CD133, and a combination thereof. In specific cases, the individual in (a), (b), or (c) is the individual from which the fibroblasts and/or dendritic cells were obtained, although not in other cases.

The neoplastic-like cells of the immunological composition may be mitotically inactivated prior to delivery to an individual. The neoplastic-like cells may be mitotically inactivated by exposure to irradiation, one or more alkylating agents, treatment with mitomycin C, or a combination thereof. In specific embodiments, the individual is provided an effective amount of one or more immune suppressive factors prior to, during, and/or after providing the immunological composition. The individual may be provided one or more agents that causes local accumulation of antigen presenting cells, including local administration of GM-CSF to the individual.

### BRIEF DESCRIPTION OF THE DRAWINGS

For a more complete understanding of the present invention, reference is now made to the following descriptions taken in conjunction with the accompanying drawings, in which:

FIG. 1 shows growth inhibition of B16 melanoma upon administration of mitotically inactivated fibroblast derived cells that have been reverted to iPS status, then differentiated along the neural lineage in the presence of mutation stimulator (hydrogen peroxide), but not in its absence. In the bar graph groupings, control is the left bar, non-mutated is the middle bar, and mutated is the right bar.

FIG. 2 shows growth inhibition of GL-261 Glioma upon administration of mitotically inactivated fibroblast derived cells that have been reverted to iPS status, then differentiated along the neural lineage in the presence of mutation stimulator (hydrogen peroxide), but not in its absence. In the bar graph groupings, control is the left bar, non-mutated is the middle bar, and mutated is the right bar.

### DETAILED DESCRIPTION

When practicing methods of the present disclosure, it should be appreciated that the present disclosure provides many applicable inventive concepts that can be embodied in a wide variety of specific contexts. The specific embodiments discussed herein are merely illustrative of specific ways to make and use the methods and compositions of the disclosure and do not limit the scope of the disclosure.

### I. Definitions

To allow for the understanding of this disclosure, a number of terms are defined below. Terms defined herein have meanings as commonly understood by a person of ordinary skill in the areas relevant to the present disclosure. The terminology herein is used to describe specific embodiments of the disclosure, but their usage does not delimit the disclosure, except as outlined in the claims.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

The term “administered” or “administering”, as used herein, refers to any method of providing a composition to an individual such that the composition has its intended effect on the individual. For example, one method of administering is by an indirect mechanism using a medical device such as, but not limited to a catheter, applicator gun, syringe, etc. A second exemplary method of administering is by a direct mechanism such as, local tissue administration, oral ingestion, transdermal patch, topical, inhalation, suppository, etc.

The term “allogeneic,” as used herein, refers to cells of the same species that differ genetically from cells of a host or recipient.

The term “autologous,” as used herein, refers to cells derived from the same subject.

The terms “antigen-presenting cells” or “APCs” are used to refer to autologous cells that express MHC Class I and/or MHC Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the disclosure and that the disclosure is not limited to the exemplary cell types described herein. APCs may be “loaded” with an antigen that is pulsed, or loaded, with antigenic peptide or recombinant peptide derived from one or more antigens. In one embodiment, a peptide is the antigen and is generally an antigenic fragment capable of inducing an immune response that is characterized by the activation of helper T cells, cytolytic T lymphocytes (cytolytic T cells or CTLs) that are directed against a malignancy or infection by a mammal. In one embodiment, the peptide includes one or more peptide fragments of an antigen that are presented by class I MHC or class II MHC molecules. The skilled artisan will recognize that peptides or protein fragments that are one or more fragments of other antigens may be used with the present disclosure, and that the disclosure is not limited to the exemplary peptides, tumor cells, cell clones, cell lines, cell supernatants, cell membranes, and/or antigens that are described herein.

The terms “dendritic cell” or “DC” refer to all DCs useful in the present disclosure, that is, DC includes various stages of differentiation, maturation and/or activation. In one embodiment of the present disclosure, the dendritic cells and responding T cells are derived from healthy volunteers. In another embodiment, the dendritic cells and T cells are derived from patients with cancer or other forms of tumor disease. In yet another embodiment, dendritic cells are used for either autologous or allogeneic application.

The term “effective amount” refers to a quantity of an antigen or epitope that is sufficient to induce or amplify an immune response against a tumor antigen, e.g., a tumor cell.

The term “vaccine” refers to compositions that affect the course of the disease by causing an effect on cells of the adaptive immune response, namely, B cells and/or T cells. The effect of vaccines can include, for example, induction of cell mediated immunity or alteration of the response of the T cell to its antigen. In some cases, the compositions of the disclosure are immunological compositions that elicit an immune response in an individual once delivered to the individual.

The term “immunological composition” as used herein refers to a composition that upon delivery to an individual invokes an immune response of any kind in the individual.

The term “immunologically effective” refers to an amount of antigen and antigen presenting cells loaded with one or more optionally heat-shocked and/or killed tumor cells that elicit a change in the immune response to prevent or treat a cancer. The amount of antigen-loaded and/or antigen-loaded APCs inserted or reinserted into the patient will vary between individuals depending on many factors. For example, different doses may be required for an effective immune response in a human with a solid tumor or a metastatic tumor.

As used herein, the term “cancer cell” refers to a cell that exhibits an abnormal morphological and/or proliferative phenotype. The cancer cell may form part of a tumor, in which case it may be defined as a tumor cell. In vitro, cancer cells are characterized by anchorage independent cell growth, loss of contact inhibition and the like, as is known to the skilled artisan. Cancer cells may be of any kind, including solid tumor cancer cells or hematopoietic or other cancer cells. The cancer may be of any tissue origin including at least brain, breast, skin, lung, stomach, colon, spleen, liver, kidney, head and neck, esophageal, intestinal, bladder, gall bladder, pituitary gland, thyroid, and so forth. As compared to normal cells, cancer cells may demonstrate abnormal new growth of tissue, e.g., a solid tumor or cells that invade surrounding tissue and metastasize to other body sites. A tumor or cancer “cell line” is generally used to describe those cells that are immortal and that may be grown in vitro. A primary cell is often used to describe a cell that is in primary culture, that is, it is freshly isolated from a patient, tissue or tumor. A cell clone will generally be used to describe a cell that has been isolated or cloned from a single cell and may or may not have been passed in in vitro culture. Examples of in vitro cancer cell lines useful for the practice of the methods of the disclosure as an antigen source include: J82, RT4, ScaBER, T24, TCCSUP, 5637 Carcinoma, SK-N-MC Neuroblastoma, SK-N-SH Neuroblastoma, SW 1088 Astrocytoma, SW 1783 Astrocytoma, U-87 MG Glioblastoma, astrocytoma, grade III, U-118 MG Glioblastoma, U-138 MG Glioblastoma, U-373 MG Glioblastoma, astrocytoma, grade III, Y79 Retinoblastoma, BT-20 Carcinoma, breast, BT-474 Ductal carcinoma, breast, MCF7 Breast adenocarcinoma, pleural effusion, MDA-MB-134-V Breast, ductal carcinoma, pleural I effusion, MDA-MD-157 Breast medulla, carcinoma, pleural effusion, MDA-MB-175-VII Breast, ductal carcinoma, pleural Effusion, MDA-MB-361 Adenocarcinoma, breast, metastasis to brain, SK-BR-3 Adenocarcinoma, breast, malignant pleural effusion, C-33 A Carcinoma, cervix, HT-3 Carcinoma, cervix, metastasis to lymph node ME-180 Epidermoid carcinoma, cervix, metastasis to omentum, MEL-175 Melanoma, MEL-290 Melanoma, HLA-A*0201 Melanoma cells, MS751 Epidermoid carcinoma, cervix, metastasis to lymph Node, SiHa Squamous carcinoma, cervix, JEG-3 Choriocarcinoma, Caco-2 Adenocarcinoma, colon HT-29 Adenocarcinoma, colon, moderately well-differentiated grade II, SK-CO-1 Adenocarcinoma, colon, ascites, HuTu 80 Adenocarcinoma, duodenum, A-253 Epidermoid carcinoma, submaxillary gland FaDu Squamous cell carcinoma, pharynx, A-498 Carcinoma, kidney, A-704 Adenocarcinoma, kidney Caki-1 Clear cell carcinoma, consistent with renal primary, metastasis to skin, Caki-2 Clear cell carcinoma, consistent with renal primary, SK-NEP-1 Wilms' tumor, pleural effusion, SW 839 Adenocarcinoma, kidney, SK-HEP-1 Adenocarcinoma, liver, ascites, A-427 Carcinoma, lung Calu-1 Epidermoid carcinoma grade III, lung, metastasis to pleura, Calu-3 Adenocarcinoma, lung, pleural effusion, Calu-6 Anaplastic carcinoma, probably lung, SK-LU-1 Adenocarcinoma, lung consistent with poorly differentiated, grade III, SK-MES-1 Squamous carcinoma, lung, pleural effusion, SW 900 Squamous cell carcinoma, lung, EB1 Burkitt lymphoma, upper maxilia, EB2 Burkitt lymphoma, ovary P3HR-1 Burkitt lymphoma, ascites, HT-144 Malignant melanoma, metastasis to subcutaneous tissue Malme-3M Malignant melanoma, metastasis to lung, RPMI-7951 Malignant melanoma, metastasis to lymph node, SK-MEL-1 Malignant melanoma, metastasis to lymphatic system, SK-MEL-2 Malignant melanoma, metastasis to skin of thigh, SK-MEL-3 Malignant melanoma, metastasis to lymph node SK-MEL-5 Malignant melanoma, metastasis to axillary node, SK-MEL-24 Malignant melanoma, metastasis to node, SK-MEL-28 Malignant melanoma, SK-MEL-31 Malignant melanoma, Caov-3 Adenocarcinoma, ovary, consistent with primary, Caov-4 Adenocarcinoma, ovary, metastasis to subserosa of fallopian tube, SK-OV-3 Adenocarcinoma, ovary, malignant ascites, SW 626 Adenocarcinoma, ovary, Capan-1 Adenocarcinoma, pancreas, metastasis to liver, Capan-2 Adenocarcinoma, pancreas, DU 145 Carcinoma, prostate, metastasis to brain, A-204 Rhabdomyosarcoma, Saos-2 Osteogenic sarcoma, primary, SK-ES-1 Anaplastic osteosarcoma versus Swing sarcoma, SK-LNS-1 Leiomyosarcoma, vulva, primary, SW 684 Fibrosarcoma, SW 872 Liposarcoma SW 982 Axilla synovial sarcoma, SW 1353 Chondrosarcoma, humerus, U-2 OS Osteogenic sarcoma, bone primary, Malme-3 Skin fibroblast, KATO III Gastric carcinoma, Cate-1B Embryonal carcinoma, testis, metastasis to lymph node, Tera-1 Embryonal carcinoma, Tera-2 Embryonal carcinoma, SW579 Thyroid carcinoma, AN3 CA Endometrial adenocarcinoma, metastatic, HEC-1-A Endometrial adenocarcinoma HEC-1-B Endometrial adenocarcinoma, SK-UT-1 Uterine, mixed mesodermal tumor, consistent with leiomyosarcomagrade III, SK-UT-1B Uterine, mixed mesodermal tumor, Sk-Me128 Melanoma SW 954 Squamous cell carcinoma, vulva, SW 962 Carcinoma, vulva, lymph node metastasis, NCI-H69 Small cell carcinoma, lung, NCI-H128 Small cell carcinoma, lung, BT-483 Ductal carcinoma, breast BT-549 Ductal carcinoma, breast, DU4475 Metastatic cutaneous nodule, breast carcinoma HBL-100 Breast, Hs 578Bst Breast, Hs 578T Ductal carcinoma, breast, MDA-MB-330 Carcinoma, breast MDA-MB-415 Adenocarcinoma, breast, MDA-MB-435s Ductal carcinoma, breast, MDA-MB-436 Adenocarcinoma, breast, MDA-MB-453 Carcinoma, breast, MDA-MB-468 Adenocarcinoma, breast T-47D Ductal carcinoma, breast, pleural effusion, Hs 766T Carcinoma, pancreas, metastatic to lymph node, Hs 746T Carcinoma, stomach, metastatic to left leg, Hs 695T Amelanotic melanoma, metastatic to lymph node, Hs 683 Glioma, Hs 294T Melanoma, metastatic to lymph node, Hs 602 Lymphoma, cervical JAR Choriocarcinoma, placenta, Hs 445 Lymphoid, Hodgkin's disease, Hs 700T Adenocarcinoma, metastatic to pelvis, H4 Neuroglioma, brain, Hs 696 Adenocarcinoma primary, unknown, metastatic to bone-sacrum, Hs 913T Fibrosarcoma, metastatic to lung, Hs 729 Rhabdomyosarcoma, left leg, FHs 738Lu Lung, normal fetus, FHs 173We Whole embryo, normal, FHs 738B1 Bladder, normal fetus NIH:OVCAR-3 Ovary, adenocarcinoma, Hs 67 Thymus, normal, RD-ES Ewing's sarcoma ChaGo K-1 Bronchogenic carcinoma, subcutaneous, metastasis, human, WERI-Rb-1 Retinoblastoma NCI-H446 Small cell carcinoma, lung, NCI-H209 Small cell carcinoma, lung, NCI-H146 Small cell carcinoma, lung, NCI-H441 Papillary adenocarcinoma, lung, NCI-H82 Small cell carcinoma, lung H9 T-cell lymphoma, NCI-H460 Large cell carcinoma, lung, NCI-H596 Adenosquamous carcinoma, lung NCI-H676B Adenocarcinoma, lung, NCI-H345 Small cell carcinoma, lung, NCI-H820 Papillary adenocarcinoma, lung, NCI-H520 Squamous cell carcinoma, lung, NCI-H661 Large cell carcinoma, lung NCI-H510A Small cell carcinoma, extra-pulmonary origin, metastatic D283 Med Medulloblastoma Daoy Medulloblastoma, D341 Med Medulloblastoma, AML-193 Acute monocyte leukemia, and MV4-11 Leukemia biphenotype.

The terms “contacted” and “exposed”, when applied to an antigen and APC, for example, are used herein to describe the process by which an antigen is placed in direct juxtaposition with the APC. To achieve antigen presentation by the APC, the antigen is provided in an amount effective to “prime” the APCs to express antigen-loaded MHC class I and/or class II antigens on the cell surface.

The term “therapeutically effective amount” refers to the amount of antigen-loaded APCs that, when administered to an animal is effective to kill cancer cells directly or indirectly within the animal. The methods and compositions of the present disclosure are equally suitable for killing a cancer cell or cells both in vitro and in vivo. When the cells to be killed are located within an animal, methods of the present disclosure may be used in conjunction or as part of a course of treatment that may also include one or more anti-neoplastic agent, e.g., chemical, irradiation, X-rays, UV-irradiation, microwaves, electronic emissions, and the like. The skilled artisan will recognize that the methods of the present disclosure may be used in conjunction with therapeutically effective amount of one or more pharmaceutical compositions, such as a DNA damaging compound, such as, Adriamycin, 5-fluorouracil, etoposide, camptothecin, actinomycin-D, mitomycin C, cisplatin and the like. However, the present methods include live cells that are going to activate other immune cells that may be affected by the DNA damaging agent. As such, any chemical and/or other course of treatment will generally be timed to maximize the adaptive immune response while at the same time aiding to kill as many cancer cells as possible.

The term “antigen-loaded dendritic cells,” “antigen-pulsed dendritic cells” and the like refer to DCs that have been contacted with an antigen, as an example in this case cancer cells that have been heat-shocked. Often, dendritic cells require a few hours, or up to a day, to process the antigen for presentation to naive and memory T-cells. It may be desirable to pulse the DC with antigen again after a day or two in order to enhance the uptake and processing of the antigen and/or provide one or more cytokines that will change the level of maturing of the DC. Once a DC has engulfed the antigen (e.g., pre-processed heat-shocked and/or killed cancer cells), it is termed an “antigen-primed DC”. Antigen-priming can be seen in DCs by immunostaining with, e.g., an antibody to the specific cancer cells used for pulsing. An antigen-loaded or pulsed DC population may be washed, concentrated, and infused directly into the patient as a type of vaccine or treatment against the pathogen or tumor cells from which the antigen originated. Generally, antigen-loaded DC are expected to interact with naive and/or memory T-lymphocytes in vivo, thus causing them to recognize and destroy cells displaying the antigen on their surfaces. In one embodiment, the antigen-loaded DC may even interact with T cells in vitro prior to reintroduction into an individual. The skilled artisan will know how to optimize the number of antigen-loaded DC per infusion, the number and the timing of infusions. For example, in one embodiment one can infuse a patient with 1-2 million antigen-pulsed cells per infusion, but fewer cells may also induce the desired immune response.

The term “individual”, as used herein, refers to a human or animal that may or may not be housed in a medical facility and may be treated as an outpatient of a medical facility. The individual may or may not be receiving one or more medical compositions from a medical practitioner and/or via the Internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The terms “subject” or “individual” may be used interchangeably and refer to any organism or animal subject that is an object of a method and/or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “prevent” or “preventing” refers to a method wherein a medical condition or onset of at least one symptom thereof is kept from occurring or is delayed in onset.

### II. General Embodiments

In one embodiment of the disclosure, patient-specific fibroblasts are used to generate autologous inducible pluripotent stem cells (iPSCs), and these (iPSCs) are utilized in preparation of one or more cancer immunological compositions, including vaccines, for the patient. In some cases, the iPSCs or derivatives and/or lysates thereof are directly used in a cancer vaccine, whereas in other embodiments derivatives of the iPSCs, components from the iPSCs, or compositions made directly or indirectly using the iPSCs or derivatives or components thereof (including lysates) are used to generate cancer vaccines. In one embodiment, the iPSCs are mutated, such as by being exposed to one or more mutagens, prior to further steps.

In one embodiment, lysate from iPSCs generated from patient-specific fibroblasts is used to pulse dendritic cells. Subsequently, the dendritic cells (DC) may be used for vaccination, in specific embodiments. In one embodiment, compounds that activate DC are utilized as adjuvants with the idea of selectively stimulating Th1 responses towards autologous patient inducible pluripotent stem cells. The DC activators may be given to DC in vitro in some embodiments, and subsequently the DC are administered to the patient. Once DC are activated by a stimulatory signal such as a TLR agonist, phagocytic activity decreases and the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [9]. In one embodiment of the disclosure, DC are cultured with autologous fibroblast-derived inducible pluripotent stem cells that have been treated in a manner to render the cells to resemble neoplastically transformed cells. Properties of cancer cells include resistance to apoptosis, lack of anchorage dependence, and ability to metastasize [10], and the neoplastically transformed cells may have one or more of these properties.

The use of DC to act as antigen presenting cells for patient-specific autologous inducible pluripotent stem cells can be realized by adapting techniques routinely used in the context of killing of tumors. Numerous animal models have demonstrated that in the context of neoplasia, DCs can bind to and engulf tumor antigens that are released from tumor cells, either alive or dying, and cross-present these antigens to T cells in tumor-draining lymph nodes. This results in the generation of tumor-specific immune responses that have been demonstrated to inhibit tumor growth or in some cases induced transferrable immunological memory. Mechanistically, DCs recognize tumors using the same molecular means that they would use to recognize apoptotic cells, or cells that are stressed. One set of signals includes molecules released from apoptotic cells, which are copiously released by tumors, and these include the nucleotides UTP and ATP, fractalkine, lipid lysophosphatidylcholine, and sphingosine 1-phosphate [11], as examples. Signals from stressed cells, such as tumor cells, include externalization of phosphatidylserine onto the outside of the cell membrane, calreticulin, avß5 integrin, CD36 and lactadherin, for example. There is some evidence that dendritic cells actively promote tumor immunogenicity in that patients with dendritic cell infiltration of tumors generally have a better prognosis [12-15].

In one embodiment of the disclosure, one or more adjuvants are used that modulate dendritic cells to stimulate antibodies that are cytotoxic, for example, complement-fixing. In one embodiment, tumor endothelial antigens are co-administered together with adjuvants that stimulate dendritic cells to program T cells in a manner to allow T cell upregulation of cytokines associated with cytotoxic antibodies, such as interferon gamma, or BLyS (interferon gamma and/or BLyS make the tumor endothelial antigens more visible to the immune system in acting as adjuvants).

In some embodiments of the disclosure, antigen-loaded DCs may be co-cultured with T-lymphocytes to produce antigen-specific T-cells. As used herein, the term “antigen-specific T-cells” refers to T-cells that proliferate upon exposure to the antigen-loaded APCs of the present disclosure, as well as to develop the ability to attack cells having the specific antigen on their surfaces. Such T-cells, e.g., cytotoxic T-cells, lyse target cells by a number of methods, e.g., releasing toxic enzymes such as granzymes and perforin onto the surface of the target cells or by effecting the entrance of these lytic enzymes into the target cell interior. Generally, cytotoxic T-cells express CD8 on their cell surface. T-cells that express the CD4 antigen, commonly known as “helper” T-cells, can also help promote specific cytotoxic activity and may also be activated by the antigen-loaded APCs of the present disclosure. In certain embodiments, the cancer cells, the APCs and even the T-cells can be derived from the same donor whose MNC yielded the DC, which can be the patient or an HLA-matched individual or obtained from the individual patient that is going to be treated. Alternatively, the cancer cells, the APCs and/or the T-cells can be allogeneic with respect to the recipient individual.

The disclosure provides means of inducing an anti-cancer response in a mammal, comprising the steps of initially “priming” the mammal by administering one or more agents that causes local accumulation of antigen presenting cells. Subsequently, tumor antigens derived from fibroblast-generated autologous inducible pluripotent stem cells are administered in the local area where one or more agents causing accumulation of antigen presenting cells is administered. A time period is allowed to pass to allow for the antigen presenting cells to traffic to the lymph nodes. Subsequently, a maturation signal, or a plurality of maturation signals, is administered to enhance the ability of the antigen presenting cells to activate adaptive immunity. In some embodiments of the disclosure, one or more activators of adaptive immunity are concurrently given, as well as suppressors of the tumor derived inhibitors (for example, checkpoint inhibitors) are administered to de-repress the immune system . . . .

In one embodiment, priming of the patient is achieved by administration of GM-CSF subcutaneously in the area in which antigen is to be injected. Various scenarios are known in the art for administration of GM-CSF prior to administration, or concurrently with administration of antigen. The practitioner of the disclosure is referred to the following publications for dosage regimens of GM-CSF and also of peptide antigens [16-27]. Subsequent to priming, one can administer tumor antigen. Various tumor antigens may be utilized, and in one particular embodiment lysed inducible pluripotent stem cells from the same patient area utilized. Means for generation of lysed cells are well known in the art and described in the following references [28-34].

One example of a method for generation of tumor lysate (which can be used for cregenerationation of protocols for dissociation of dedifferentiated fibroblasts) involves obtaining frozen autologous samples that are placed in Hanks buffered saline solution (HBSS) and gentamycin 50 μg/ml followed by homogenization by a glass homogenizer. After repeated freezing and thawing, particle-containing samples are selected and frozen in aliquots after radiation with 25 kGy. Quality assessment for sterility and endotoxin content is performed before freezing. Cell lysates are subsequently administered into the patient in a preferred manner subcutaneously at the local areas where DC priming was initiated. In a specific embodiment, after 12-72 hours, the patient is subsequently administered with one or more agents capable of inducing maturation of DC. Agents useful for maturing DCs in the disclosure, in a particular embodiment, include BCG and HMGB1 peptide. Other useful agents include the following: a) histone DNA; b) imiqimod; c) beta-glucan; d) hsp65; e) hsp90; f) HMGB-1; g) lipopolysaccharide; h) Pam3CSK4; i) Poly I: Poly C; j) Flagellin; k) MALP-2; 1) Imidazoquinoline; m) Resiquimod; n) CpG oligonucleotides; o) zymosan; p) peptidoglycan; q) lipoteichoic acid; r) lipoprotein from gram-positive bacteria; s) lipoarabinomannan from mycobacteria; t) Polyadenylic-polyuridylic acid; u) monophosphoryl lipid A; v) single stranded RNA; w) double stranded RNA; x) 852A; y) rintatolimod; z) Gardiquimod; and aa) lipopolysaccharide peptides. The procedure is performed in a particular embodiment with the administration of Indoleamine-pyrrole 2,3-dioxygenase (IDO) silencing siRNA or shRNA containing the effector sequences a) UUAUAAUGACUGGAUGUUC (SEQ ID NO:1); b) GUCUGGUGUAUGAAGGGUU (SEQ ID NO:2); c) CUCCUAUUUUGGUUUAUGC (SEQ ID NO:3) and d) GCAGCGUCUUUCAGUGCUU (SEQ ID NO:4). siRNA or shRNA may be administered through various modalities including biodegradable matrices, pressure gradients or viral transfect. In another embodiment, autologous dendritic cells are generated and IDO is silenced, prior to, concurrent with or subsequent to silencing, and the dendritic cells are pulsed with tumor antigen and administered systemically.

Culture of dendritic cells is well known in the art, for example, U.S. Pat. No. 6,936,468, issued to Robbins, et al., for the use of tolerogenic dendritic cells for enhancing tolerogenicity in a host and methods for making the same. Although the current disclosure aims to reduce tolerogenesis, the essential means of dendritic cell generation are disclosed in the patent. U.S. Pat. No. 6,734,014, issued to Hwu, et al., for methods and compositions for transforming dendritic cells and activating T cells. Briefly, recombinant dendritic cells are made by transforming a stem cell and differentiating the stem cell into a dendritic cell. The resulting dendritic cell is said to be an antigen presenting cell that activates T cells against MHC class I-antigen targets. Antigens for use in dendritic cell loading are taught in, e.g., U.S. Pat. No. 6,602,709, issued to Albert, et al. This patent teaches methods for use of apoptotic cells to deliver antigen to dendritic cells for induction or tolerization of T cells. The methods and compositions are said to be useful for delivering antigens to dendritic cells that are useful for inducing antigen-specific cytotoxic T lymphocytes and T helper cells. The disclosure includes assays for evaluating the activity of cytotoxic T lymphocytes. The antigens targeted to dendritic cells are apoptotic cells that may also be modified to express non-native antigens for presentation to the dendritic cells. The dendritic cells are said to be primed by the apoptotic cells (and fragments thereof) capable of processing and presenting the processed antigen and inducing cytotoxic T lymphocyte activity or may also be used in vaccine therapies. U.S. Pat. No. 6,455,299, issued to Steinman, et al., teaches methods of use for viral vectors to deliver antigen to dendritic cells. Methods and compositions are said to be useful for delivering antigens to dendritic cells, which are then useful for inducing T antigen specific cytotoxic T lymphocytes. The disclosure provides assays for evaluating the activity of cytotoxic T lymphocytes. Antigens are provided to dendritic cells using a viral vector such as influenza virus that may be modified to express non-native antigens for presentation to the dendritic cells. The dendritic cells are infected with the vector and are said to be capable of presenting the antigen and inducing cytotoxic T lymphocyte activity or may also be used as vaccines.

In some embodiments of the disclosure, one or more adjuvants are administered together with irradiated autologous patient-derived inducible pluripotent cells or are administered with dendritic cells, which are further injected in vivo. Adjuvants useful for the practice of methods of the disclosure are selected from the group consisting of Cationic liposome-DNA complex JVRS-100, aluminum hydroxide, aluminum phosphate vaccine, aluminum potassium sulfate adjuvant, Alhydrogel, ISCOM(s), Freund's Complete Adjuvant, Freund's Incomplete Adjuvant, CpG DNA Vaccine Adjuvant, Cholera toxin, Cholera toxin B subunit liposomes, Saponin, DDA, Squalene-based Adjuvants, Etx B subunit, IL-12, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, thalidomide, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1(3, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and Matrix-S.

In another embodiment, the disclosure encompasses the pulsing of DC with extracts from fibroblast-derived inducible pluripotent stem cells, and the extract may comprise exosomes, lysate, and/or conditioned media from the fibroblast-derived inducible pluripotent stem cells. DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment, approximately 150 mL of blood is processed. The leukopheresis product may be subsequently used for initiation of dendritic cell culture. In order to generate peripheral blood mononuclear cells from leukopheresis product, mononuclear cells are isolated by the Ficoll-Hypaque densitys gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with patient specific fibroblast derived inducible pluripotent stem cells. Pulsing may be performed by incubation of lysates with dendritic cells, or may be generated by fusion of immature dendritic cells with autologous fibroblast derived inducible pluripotent stem cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests may be performed at the beginning of the culture, on the fifth day and at the time of cell freezing for further use or prior to release of the dendritic cells. Administration of autologous fibroblast-derived pluripotent cell lysate pulsed dendritic cells may be utilized as a polyvalent vaccine, whereas subsequent to administration antibody or T cell responses are assessed for induction of antigen specificity, peptides corresponding to immune response stimulated are used for further immunization to focus the immune response.

In some embodiments, culture of the immune effectors cells is performed after extracting from an individual that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the disclosure, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, TN-gamma, transforming growth factor (TGF)-beta, IL-15, IL-7, IFN-alpha, IL-12, CD40L, and IL-27. From the viewpoint of enhancing cellular immunity, particularly suitably, IL-2, IFN-gamma, or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current disclosure to optimize the cellular product based on other means of assessing T cell activity, for example, the function enhancement of the T cell in the method of the present disclosure can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the disclosure may be assessed in a living body before first administration of the T cells with enhanced function of the present disclosure, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects.

Within the context of the disclosure, teachings are provided to amplify an antigen specific immune response following immunization with a polyvalent autologous fibroblast derived inducible pluripotent stem cell vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T_{H1}-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

Embodiments of the disclosure include personalized cancer vaccines generated to possess characteristics of a patient and the cells of said patient's tumor, wherein said cancer vaccine is generated through the steps of: a) obtaining a fibroblast population from said cancer patient; b) dedifferentiating the fibroblasts into pluripotent-like cells; c) differentiating said pluripotent-like cells along the lineage of cells of which said patient cancer is comprised of; d) exposing said cell population to one or more mutagenic agents in order to replicate the oncogenic processes that occurred in said cancer patient, thereby producing mutated cells; e) growing said mutated cells in vitro alone or using feeder cells in a manner to expand “cancer stem cell”-like cells and f) utilizing said cells as an antigenic source for vaccination. In particular embodiments, the vaccine is utilized prophylactically and/or is utilized therapeutically.

The fibroblasts may be extracted from any tissue including a) skin; b) adipose; c) hair follicle; d) bone marrow; e) omentum; f) endometrium; and/or g) peripheral blood, for example. In specific embodiments, the fibroblasts are dedifferentiated into pluripotent-like cells by treatment with an activity capable of inducing biological effects similar to the effects of NANOG, OCT-4, and SOX-2 transfection. The biological effects that may be similar to the effects of NANOG, OCT-4, and SOX-2 transfection include the generation of inducible pluripotent stem cells. In specific embodiments, the inducible pluripotent stem cell properties include the ability of the cells to differentiate into cells of the mesodermal, endodermal and/or ectodermal lineages. In specific cases, the inducible pluripotent stem cells are capable of proliferating in vitro beyond the Hayflick limit.

In particular embodiments, the dedifferentiated fibroblast is differentiated into tissue associated with a cancer of origin of the patient through culture of lineage specific differentiation factors. The dedifferentiated cell may or may not be utilized as a “tissue nonspecific” cancer vaccine. The dedifferentiated cells may be treated with one or more mutagenic agents in tissue culture to endow a neoplastic phenotype. The dedifferentiated cells may be treated with one or more mutagenic agents in tissue culture during differentiation to endow a neoplastic phenotype.

Cells may be mitotically inactivated before administration, and the mitotic inactivation may be performed by irradiation, by one or more alkylating agents, and/or by treatment with mitomycin C. In particular embodiments, inhibition of an immune suppressive molecules is performed before, during, and/or after administration of the personalized cancer vaccine. Examples of immune suppressive molecules include IL-10, IL-6, PGE-2, one or more tryptophan metabolites (examples including kynurenine, putrescine, and/or spermine); and/or one or more arginine metabolites (examples including ornithine and/or polyamine).

### EXAMPLE

The following example is included to demonstrate particular embodiments of the disclosure. It should be appreciated by those of skill in the art that the techniques disclosed in the examples that follow represent techniques discovered by the inventor to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Induction of Tumor Immunity in Melanoma

The murine embryonic fibroblast-derived iPS cell line iPS-MEF-Ng-20D-17 was maintained in Dulbecco's modified Eagle's medium (DMEM) containing 15% embryonic stem screened fetal bovine serum (FBS) (Thermo Scientific, Yokohama, Japan), 2 mM 1-glutamine (Thermo Scientific), 100 U/ml of penicillin, 100 mg/ml of streptomycin (Life Technologies Co., Carlsbad, Calif.), nonessential amino acids (Life Technologies) and 50 μM of 2-mercaptoethanol (2-ME) (Life Technologies) on feeder cell layers of mitomycin C-treated murine SNL76/7 cells (European Collection of Cell Cultures, London, UK). Cells were treated with the procedure of dissociating into small aggregates using collagenase (Invitrogen) and plating on non-adhesive plastic in human ESC media (DMEM/F12 or knock-out DMEM, 0.1 mM NEAA, 0.1 mM beta-mercaptoethanol, 2 mM L-glutamine, 15% or 20% (KSR); all from Invitrogen) without FGF2 to induce differentiation.

Media was changed every second to third day, and 5-7-day-old floating aggregates were plated on tissue culture plates coated with 0.1 mg/ml poly-L-ornithine (Sigma). Neural rosette structures started to emerge about one week after plating. Rosettes were carefully picked every second day between one and two weeks post plating. Picking was performed with a needle, and picked clusters were inspected under the microscope for purity before transfer to a non-adhesive culture plate containing DMEM/F12, 2 mM L-glutamine, 1.6 g/l glucose, 0.1 mg/ml Penicillin/Streptomycin and N2 supplement (1:100; Invitrogen). After 2-5 days floating aggregates were dissociated in trypsin for 5-10 minutes. Trypsin activity was inhibited with trypsin inhibitor before cells were spun down for 5 minutes at 300 g. Media was carefully aspirated to avoid any remaining trypsin, and cells were plated onto poly-L-ornithine and 10 μg/ml laminin (Sigma) coated plates into the same media supplemented with 10 ng/ml FGF2, 10 ng/ml EGF (both from R&D systems) and B27 (1 μl/ml, Invitrogen). Cells were passaged at a ratio of 1:3 every second to third day using trypsin. Neuronal differentiation was induced by removing the growth factors FGF2 and EGF from the media and culturing the cells in a 1:1 ratio mixture of Neurobasal media supplemented with B27 (1:50, Invitrogen) and DMEM/F12 media supplemented with N2 (1:100); 300 ng/ml cAMP was added to the differentiation media. During this type some cells were exposed to hydrogen peroxide 1/1,0000 v/v. The fate of the differentiated cells was quantitatively assessed by counting 250-650 cells in nine 20× microscope fields from 3-4 experiments. Living cells were mitotically inactivated by culture in 0.5 uM of Mitomycin C for 2 hours.

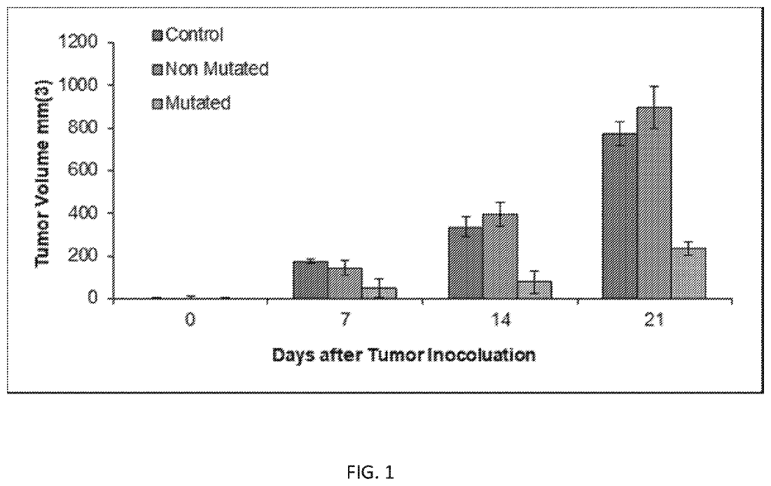

Cells were inoculated at a concentration of 500,000 cells per C57/BL6 mouse bearing B16 melanoma, inoculated in the flank at a concentration of 500,000 cells per mouse. In FIG. 1, the “non-mutated” are cells not treated with hydrogen peroxide, whereas “mutated” were treated. The data demonstrate that the growth of B16 melanoma is inhibited by administration of mitotically inactivated fibroblast derived cells that have been reverted to iPS status, then differentiated along the neural lineage in the presence of mutation stimulator (hydrogen peroxide), but not in its absence.

### Example 2

### Induction of Tumor Immunity in Glioma

The murine embryonic fibroblast-derived iPS cell line iPS-MEF-Ng-20D-17 was maintained in Dulbecco's modified Eagle's medium (DMEM) containing 15% embryonic stem screened fetal bovine serum (FBS) (Thermo Scientific, Yokohama, Japan), 2 mM 1-glutamine (Thermo Scientific), 100 U/ml of penicillin, 100 mg/ml of streptomycin (Life Technologies Co., Carlsbad, Calif.), nonessential amino acids (Life Technologies) and 50 μM of 2-mercaptoethanol (2-ME) (Life Technologies) on feeder cell layers of mitomycin C-treated murine SNL76/7 cells (European Collection of Cell Cultures, London, UK). Cells were treated with the procedure of dissociating into small aggregates using collagenase (Invitrogen) and plated on non-adhesive plastic in human ESC media (DMEM/F12 or knock-out DMEM, 0.1 mM NEAA, 0.1 mM beta-mercaptoethanol, 2 mM L-glutamine, 15% or 20% (KSR); all from Invitrogen) without FGF2 to induce differentiation.

Media was changed every second to third day, and 5-7-day-old floating aggregates were plated on tissue culture plates coated with 0.1 mg/ml poly-L-ornithine (Sigma). Neural rosette structures started to emerge about one week after plating. Rosettes were carefully picked every second day between one and two weeks post plating. Picking was performed with a needle, and picked clusters were inspected under the microscope for purity before transfer to a non-adhesive culture plate containing DMEM/F12, 2 mM L-glutamine, 1.6 g/l glucose, 0.1 mg/ml Penicillin/Streptomycin and N2 supplement (1:100; Invitrogen). After 2-5 days floating aggregates were dissociated in trypsin for 5-10 minutes. Trypsin activity was inhibited with trypsin inhibitor before cells were spun down for 5 minutes at 300 g. Media was carefully aspirated to avoid any remaining trypsin, and cells were plated onto poly-L-ornithine and 10 μg/ml laminin (Sigma) coated plates into the same media supplemented with 10 ng/ml FGF2, 10 ng/ml EGF (both from R&D systems) and B27 (1 μl/ml, Invitrogen). Cells were passaged at a ratio of 1:3 every second to third day using trypsin. Neuronal differentiation was induced by removing the growth factors FGF2 and EGF from the media and culturing the cells in a 1:1 ratio mixture of Neurobasal media supplemented with B27 (1:50, Invitrogen) and DMEM/F12 media supplemented with N2 (1:100); 300 ng/ml cAMP was added to the differentiation media. During this type some cells were exposed to hydrogen peroxide 1/1,0000 v/v. The fate of the differentiated cells was quantitatively assessed by counting 250-650 cells in nine 20× microscope fields from 3-4 experiments. Living cells were mitotically inactivated by culture in 0.5 uM of Mitomycin C for 2 hours.

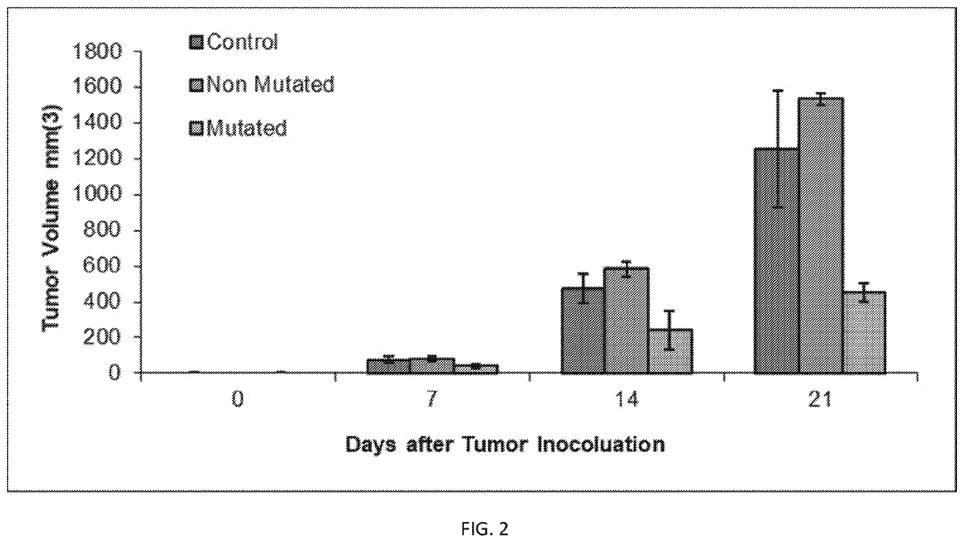

Cells were inoculated at a concentration of 500,000 cells per C57/BL6 mouse bearing GL-261 Glioma, inoculated in the flank at a concentration of 500,000 cells per mouse. In FIG. 2, the “non-mutated” are cells not treated with hydrogen peroxide, whereas “mutated” were treated. The data demonstrate that the growth of GL-261 Glioma is inhibited by administration of mitotically inactivated fibroblast derived cells that have been reverted to iPS status, then differentiated along the neural lineage in the presence of mutation stimulator (hydrogen peroxide), but not in its absence.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
1. Flexner S, Jobling J W. Proc Soc Exp Biol Med. 1907. p. 461.
2. Clerici, M., E. Clerici, and G. M. Shearer, The tumor enhancement phenomenon: reinterpretation from a Th1/Th2 perspective. J Natl Cancer Inst, 1996. 88(7): p. 461-2.
3. Kaliss, N., Immunological enhancement of tumor homografts in mice: a review. Cancer Res, 1958. 18(9): p. 992-1003.
4. Prehn, R. T., The immune reaction as a stimulator of tumor growth. Science, 1972. 176(4031): p. 170-1.
5. Alexandrescu, D. T., et al., Immunotherapy for melanoma: current status and perspectives. J Immunother, 2010. 33(6): p. 570-90.
6. Wang, Y., et al., Unique molecular events during reprogramming of human somatic cells to induced pluripotent stem cells (iPSCs) at naive state. Elife, 2018. 7.
7. Wattanapanitch, M., et al., Dual small-molecule targeting of SMAD signaling stimulates human induced pluripotent stem cells toward neural lineages. PLoS One, 2014. 9(9): p. e106952.
8. Zhang, Z. and W. S. Wu, Sodium butyrate promotes generation of human induced pluripotent stem cells through induction of the miR302/367 cluster. Stem Cells Dev, 2013. 22(16): p. 2268-77.
9. Itano, A. A. and M. K. Jenkins, Antigen presentation to naive CD4 T cells in the lymph node. Nat Immunol, 2003. 4(8): p. 733-9.
10. O'Shannessy, D. J., et al., Influence of tumor microenvironment on prognosis in colorectal cancer: Tissue architecture-dependent signature of endosialin (TEM-1) and associated proteins. Oncotarget, 2014. 5(12): p. 3983-95.
11. Ravichandran, K. S., Beginnings of a good apoptotic meal: the find-me and eat-me signaling pathways. Immunity, 2011. 35(4): p. 445-55.
12. Hu, M., et al., Decreased intratumoral Foxp3 Tregs and increased dendritic cell density by neoadjuvant chemotherapy associated with favorable prognosis in advanced gastric cancer. Int J Clin Exp Pathol, 2014. 7(8): p. 4685-94.
13. Ayari, C., et al., High level of mature tumor-infiltrating dendritic cells predicts progression to muscle invasion in bladder cancer. Hum Pathol, 2013. 44(8): p. 1630-7.
14. Liska, V., et al., Infiltration of colorectal carcinoma by S100+ dendritic cells and CD57+ lymphocytes as independent prognostic factors after radical surgical treatment. Anticancer Res, 2012. 32(5): p. 2129-32.
15. Ayari, C., et al., Bladder tumor infiltrating mature dendritic cells and macrophages as predictors of response to bacillus Calmette-Guerin immunotherapy. Eur Urol, 2009. 55(6): p. 1386-95.
16. Middleton, G., et al., Gemcitabine and capecitabine with or without telomerase peptide vaccine GV1001 in patients with locally advanced or metastatic pancreatic cancer (TeloVac): an open-label, randomised, phase 3 trial. Lancet Oncol, 2014. 15(8): p. 829-40.
17. Mittendorf, E. A., et al., Final report of the phase I/II clinical trial of the E75 (nelipepimut-S) vaccine with booster inoculations to prevent disease recurrence in high-risk breast cancer patients. Ann Oncol, 2014. 25(9): p. 1735-42.
18. Rahma, O. E., et al., The immunological and clinical effects of mutated ras peptide vaccine in combination with IL-2, GM-CSF, or both in patients with solid tumors. J Transl Med, 2014. 12: p. 55.
19. Clancy-Thompson, E., et al., Peptide vaccination in Montanide adjuvant induces and GM-CSF increases CXCR3 and cutaneous lymphocyte antigen expression by tumor antigen-specific CD8 T cells. Cancer Immunol Res, 2013. 1(5): p. 332-9.
20. Sonpavde, G., et al., HLA-restricted NY-ESO-1 peptide immunotherapy for metastatic castration resistant prostate cancer. Invest New Drugs, 2014. 32(2): p. 235-42.
21. Geynisman, D. M., et al., A randomized pilot phase I study of modified carcinoembryonic antigen (CEA) peptide (CAP1-6D)/montanide/GM-CSF-vaccine in patients with pancreatic adenocarcinoma. J Immunother Cancer, 2013. 1: p. 8.
22. Tarhini, A. A., et al., Differing patterns of circulating regulatory T cells and myeloid-derived suppressor cells in metastatic melanoma patients receiving anti-CTLA4 antibody and interferon-alpha or TLR-9 agonist and GM-CSF with peptide vaccination. J Immunother, 2012. 35(9): p. 702-10.
23. Walter, S., et al., Multipeptide immune response to cancer vaccine IMA901 after single-dose cyclophosphamide associates with longer patient survival. Nat Med, 2012. 18(8): p. 1254-61.
24. Ohno, S., et al., Phase I trial of Wilms' Tumor 1 (WT1) peptide vaccine with GM-CSF or CpG in patients with solid malignancy. Anticancer Res, 2012. 32(6): p. 2263-9.
25. Tarhini, A. A., et al., Safety and immunogenicity of vaccination with MART-1 (26-35, 27L), gp100 (209-217, 210M), and tyrosinase (368-376, 370D) in adjuvant with PF-3512676 and GM-CSF in metastatic melanoma. J Immunother, 2012. 35(4): p. 359-66.
26. Schaefer, C., et al., Function but not phenotype of melanoma peptide-specific CD8(+) T cells correlate with survival in a multiepitope peptide vaccine trial (ECOG 1696). Int J Cancer, 2012. 131(4): p. 874-84.
27. Block, M. S., et al., Pilot study of granulocyte-macrophage colony-stimulating factor and interleukin-2 as immune adjuvants for a melanoma peptide vaccine. Melanoma Res, 2011. 21(5): p. 438-45.
28. Bapsy, P. P., et al., Open-label, multi-center, non-randomized, single-arm study to evaluate the safety and efficacy of dendritic cell immunotherapy in patients with refractory solid malignancies, on supportive care. Cytotherapy, 2014. 16(2): p. 234-44.
29. Reyes, D., et al., Tumour cell lysate-loaded dendritic cell vaccine induces biochemical and memory immune response in castration-resistant prostate cancer patients. Br J Cancer, 2013. 109(6): p. 1488-97.
30. Kamigaki, T., et al., Immunotherapy of autologous tumor lysate-loaded dendritic cell vaccines by a closed-flow electroporation system for solid tumors. Anticancer Res, 2013. 33(7): p. 2971-6.
31. Florcken, A., et al., Allogeneic partially HLA-matched dendritic cells pulsed with autologous tumor cell lysate as a vaccine in metastatic renal cell cancer: a clinical phase I/II study. Hum Vaccin Immunother, 2013. 9(6): p. 1217-27.
32. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
33. Alfaro, C., et al., Pilot clinical trial of type 1 dendritic cells loaded with autologous tumor lysates combined with GM-CSF, pegylated IFN, and cyclophosphamide for metastatic cancer patients. J Immunol, 2011. 187(11): p. 6130-42.
34. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
U.S. Pat. No. 6,455,299
U.S. Pat. No. 6,602,709
U.S. Pat. No. 6,734,014
U.S. Pat. No. 6,936,468

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of preparing an immunological composition for cancer for an individual, comprising the steps of:
(a) generating pluripotent-like cells from fibroblasts; and
(b) performing one or both of the following:
(1) exposing the pluripotent-like cells to one or more differentiation factors that differentiate the pluripotent-like cells to neoplastic-like cells; and/or
(2) exposing the pluripotent-like cells to one or more mutagenic agents, thereby producing neoplastic-like cells;
wherein the neoplastic-like cells and/or derivatives and/or lysates thereof are comprised in the immunological composition and/or are used as an antigenic source for antigen presenting cells for the individual.

2. The method of claim 1, wherein the neoplastic-like cells are expanded in culture prior to a use.

3. The method of claim 2, wherein the culture comprises feeder cells.

4. The method of claim 3, wherein the feeder cells are fibroblast cells.

5. The method of claim 1, wherein the pluripotent-like cells or the neoplastic-like cells are differentiated into cells having one or more markers of the same tissue as the tissue of the cancer.

6. The method of claim 1, wherein the neoplastic-like cells and/or derivatives and/or lysates thereof are exposed to dendritic cells to produce antigen-loaded dendritic cells.

7. The method of claim 6, wherein the exposure occurs ex vivo.

8. The method of claim 6, wherein the exposure of the lysate and/or cell fragments to dendritic cells occurs in the presence of one or more dendritic cell activators.

9. The method of claim 5, wherein the antigen-loaded dendritic cells are co-cultured with T lymphocytes to produce antigen-specific T cells.

10. The method of claim 5, wherein the dendritic cells and the fibroblast cells are from the same individual.

11. The method of claim 1, wherein the pluripotent-like cells are generated from fibroblasts upon exposure of the fibroblasts to NANOG; OCT-4; SOX-2; stem cells and/or cytoplasm from stem cells; one or more histone deacetylase inhibitors; one or more DNA methyltransferase inhibitors; one or more histone modifiers; umbilical cord blood serum; one or more GSK-3 inhibitors; or a combination thereof.

12. The method of claim 1, wherein the pluripotent-like cells are generated from fibroblasts upon exposure of the fibroblasts to reversin, cord blood serum, lithium, a GSK-3 inhibitor, resveratrol, pterostilbene, selenium, (−)-epigallocatechin-3-gallate (EGCG), valproic acid and/or salts of valproic acid, or a combination thereof

13. The method of claim 11, wherein the histone deacetylase inhibitor is selected from the group consisting of: a) valproic acid; b) sodium phenylbutyrate; c) butyrate; d) trichostatin A; and e) a combination thereof.

14. The method of claim 11, wherein the DNA methyltransferase inhibitor is selected from the group consisting of a) decitabine; b) 5-azacytidine; c) Zebularine; d) RG-108; e) procaine hydrochloride; f) Procainamide hydrochloride; g) Hydralazine hydrochloride; h) Epigallocatechin gallate; i) Chlorogenic acid; j) Caffeic acid; and h) a combination thereof.

15. The method of claim 1, wherein the de-differentiated fibroblasts are exposed to 2%-8%, 2%-7%, 2%-6%, 2%-5%, 2%-4%, 2%-3%, 3%-8%, 3%-7%, 3%-6%, 3%-5%, 3%-4%, 4%-8%, 4%-7%, 4%-6%, 4%-5%, 5%-8%, 5%-7%, 5%-6%, 6%-8%, 6%-7%, or 7%-8% oxygen.

16. The method of claim 1, wherein one or more of the following occurs:
(a) an effective amount of the immunological composition is provided to an individual;
(b) an effective amount of antigen-loaded dendritic cells produced upon exposure of dendritic cells to lysate and/or cell fragments from the neoplastic-like cells are provided to an individual;
(c) an effective amount of antigen-specific T cells produced upon exposure of the antigen-loaded dendritic cells to T lymphocytes are provided to an individual.

17. The method of claim 16, wherein one or more adjuvants are also provided to the individual in (a), (b), or (c).

18. The method of claim 17, wherein the one or more adjuvants comprise one or more toll like receptors.

19. The method of claim 16, wherein one or more tumor endothelial antigens are provided to the individual in (a), (b), or (c).

20. The method of claim 19, wherein the one or more tumor endothelial antigens is selected from the group consisting of Flt-3 ligand, TEM-1, NANOG, SOX2, CD133, and a combination thereof.

21. The method of claim 16, wherein the individual in (a), (b), or (c) is the individual from which the fibroblasts and/or dendritic cells were obtained.

22. The method of claim 1, wherein the neoplastic-like cells of the immunological composition are mitotically inactivated prior to delivery to an individual.

23. The method of claim 22, wherein the neoplastic-like cells are mitotically inactivated by exposure to irradiation, one or more alkylating agents, treatment with mitomycin C, or a combination thereof.

24. The method of claim 16, wherein the individual is provided an effective amount of one or more immune suppressive factors prior to, during, and/or after providing the immunological composition.

25. The method of claim 16, wherein the individual is provided one or more agents that causes local accumulation of antigen presenting cells.

26. The method of claim 25, further defined as local administration of GM-CSF to the individual.
